(19) United States
(12) Patent Application Publication
(10) Pub. No.: US 2009/0156694 A1
(43) Pub. Date: Jun. 18, 2009

(54) LONG-CHAIN POLYUNSATURATED FATTY ACID OIL AND COMPOSITIONS AND PREPARATION PROCESS FOR THE SAME

(75) Inventors: Raymond Bertholet, Blonay (CH); Junkuan Wang, Lonay (CH); Pierre Lambelet, Saint-Legier (CH); Heribert Watzke, Pully (CH); Zdenek Kratky, New Milford, CT (US)

Correspondence Address: K&L Gates LLP, P.O. Box 1135, CHICAGO, IL, 60690, US

(73) Assignee: Nestec S.A., Vevey (CH)

(21) Appl. No.: 12/338,643

(22) Filed: Dec. 18, 2008

Related U.S. Application Data
(63) Continuation of application No. 10/658,522, filed on Sep. 8, 2003.
Application No. 10/658,522 is a continuation of application No. PCT/EP02/02333, filed on Mar. 7, 2002.

(30) Foreign Application Priority Data
Mar. 9, 2001 (EP) 01105960.7

(51) Int. Cl. A61K 47/12; A61P 43/00; A61Q 90/00; A23D 9/00; A23D 9/04; A23K 1/16

(52) U.S. Cl. 514/784; 426/601; 426/33; 426/61

(57) ABSTRACT

Stable oil containing long-chain polyunsaturated essential fatty acids (LC-PUFAs) in the form of triacylglycerols, in particular arachidonic acid (ARA), dihomogammalinolenic acid (DHGLA), docosahexaenoic acid (DHA) or eicosapentaenoic acid (EPA). Such an oil may be prepared by bringing a carrier oil into contact with a biomass obtained from the culture of a microorganism, in particular a fungus or a microalga containing the acids ARA, DHGLA, DHA or EPA. These oils can be incorporated into the composition of a foodstuff, of a cosmetic or pharmaceutical product.

### CROSS-REFERENCE TO RELATED APPLICATIONS

The present application is a continuation of International application PCT/EP02/02333, filed Mar. 7, 2002 the entire content of which is expressly incorporated herein by reference thereto.

### BACKGROUND

The present invention relates to oils that can be used as an ingredient which is a source of long-chain polyunsaturated essential fatty acids (LC-PUFA) in a foodstuff, in a nutritional supplement, in a cosmetic or pharmaceutical composition.

An oil containing LC-PUFAs such as for example arachidonic acid (ARA), docosahexaenoic acid (DHA) or dihomogammalinolenic acid (DHGLA) may be obtained from a biomass fermentation broth. To obtain the oil from the biomass, methods of extraction with organic solvent, for example hexane, or with supercritical fluids, have been used. Generally, the oil has been extracted from biomass by percolation of the dried biomass with hexane. Such a process of extraction with organic solvent(s) is described, for example, in WO97-37032, in WO97-43362 or in the publication Journal of Dispersion Science and Technology, 10, 561-579, 1989 “Biotechnological processes for the production of PUFAs”.

This technique has various disadvantages:
During the stages of extraction with hot solvent or of distillation of the solvent, the LC-PUFAs may undergo degradation in contact with oxygen.
The complete removal of the solvent contained in the oil or in the residual biomass requires a heat treatment at high temperature.
Moreover, the solvent, such as hexane, is capable of dissolving nontriacylglycerol constituents of the biomass which in fact constitute impurities. The crude oil obtained after evaporation of the solvent should further undergo several refining stages comprising degumming, neutralization with alkali, decolorization, dewaxing and deodorization with the aim of at least partially removing the impurities. This means that the highly unsaturated oil is exposed to conditions stimulating physicochemical reactions which affect its quality. For example, the decolorization agents create a system of conjugated double bonds and form degradation products by chemical reaction with the oxidized glycerides.

The present invention seeks to avoid the disadvantages of the prior art, by providing a stable oil containing one or more polyunsaturated fatty acids derived from biomass in the form of triacylglycerols in the purified state and which has undergone minimum degradation.

### SUMMARY OF THE INVENTION

The present invention relates to a stable oil containing one or more LC-PUFAs in the form of triacylglycerols, in particular arachidonic acid (ARA), dihomogammalinolenic acid (DHGLA), docosahexaenoic acid (DHA) or eicosapentaenoic acid (EPA).

The invention also relates to a process for preparing such an oil by bringing a carrier oil into contact with a biomass obtained from the culture of a microorganism, in particular a fungus or a microalga containing the acids ARA, DHGLA, DHA or EPA.

Preferably, the oil does not contain more than 10% by weight of polyunsaturated fatty acids. As a result, the oil is much less sensitive to oxidation during its production, which is not the case for the oils containing LC-PUFAs of the prior art.

According to a main aspect of the invention, it is a crucial qualitative advantage to provide a novel oil containing LC-PUFAs in the form of triacylglycerols.

According to another aspect of the invention, the preceding oil can be incorporated into a foodstuff, a cosmetic or pharmaceutical product, a nutritional supplement or an animal feed. A preferred product is an animal feed, in particular for pets, that contains an oil obtainable from the biomass residue derived from the process of the invention.

### DETAILED DESCRIPTION OF THE PREFERRED EMBODIMENTS

The conversion is carried out by bringing the carrier oil into contact with a biomass containing the LC-PUFAs. The oil is suitable for application in foodstuffs, in particular infant formulas or for use as a nutritional supplement. It may also be used in cosmetic or pharmaceutical products. Furthermore, the biomass residue obtained is also a product of the process which may be upgraded directly without subsequent treatment, for example as animal feed, in particular for pets.

The preparation of such an oil may take place by simply mixing the carrier oil with the dried biomass and subsequently separating the oil from the non-lipid solids by pressing.

In order to increase the yield of LC-PUFA obtained it is preferable to reduce the sizes of the particles of dry biomass in order to break the walls of the cells of microorganisms and to thereby increase the surface area of contact between the oil and the biomass. This may be appropriately carried out using various methods, for example:

the biomass may be ground in the presence of the carrier oil;

the biomass may be laminated before mixing it with the carrier oil;

the biomass may be treated at high pressure in the presence of the carrier oil, and then the oil obtained may be separated from the biomass by pressing and final filtration;

the biomass may be treated with enzymes capable of degrading the walls of the cells.

Because the carrier is an oil, the oil obtained after contact with the biomass has a minimum content of phospholipids, free fatty acids, pigments, polymers and other substances obtained or derived from the biomass which are not triacylglycerols. This means that the process according to the invention constitutes a selective method for preparing a stable purified oil containing LC-PUFAS. It is not necessary to purify the unsaturated oil containing the LC-PUFAs by the aggressive and cumbersome methods used prior to the invention such as the stages of degumming, neutralization, dewaxing and decolorization.

According to the invention the oil is solely subjected to a stage of deodorization, for example by steam distillation or molecular distillation at a relatively low temperature. The result is that the oil contains a particularly small quantity of trans fatty acids.

The process does not use an organic solvent and, since the operation is preferably carried out under a nitrogen atmosphere and in the presence of tocopherols which are naturally present or which are added to the carrier oil, in an amount sufficient to protect the LC-PUFAs from oxidative degradation during the preparation process.

In addition to the quality of the oil obtained, another advantage of the process is that the biomass residue is not contaminated with an organic solvent and may thus be directly upgraded, without subsequent treatment, for example in animal feed, in particular for pets.

The detailed description of the process which follows is targeted at the preparation of an oil containing ARA, taken by way of non-limiting example. The working conditions for transferring other LC-PUFAs to a carrier oil from appropriate biomasses, for example for DHA or DHGLA, are very similar.

The oil is obtained by mixing the carrier oil with the dry biomass and separating the oil from the solid components by pressing. To increase the level of incorporation of ARA, it is desirable to break the microbial cells by high-pressure treatments, by enzymatic processes or to reduce the sizes of the dry particles of biomass by grinding or laminating.

The grinding step used may be one of many techniques known in the prior art, for example, the biomass may be laminated, preferably at low temperature, and then it may be mixed with the carrier oil. As a variant, the biomass may be ground in the presence of the carrier oil. In order to minimize as much as possible damage to the ARA, the grinding conditions should be gentle. In this regard, grinding the biomass in the presence of the carrier oil under gentle conditions, at a moderate temperature, and under an inert atmosphere, for example under a nitrogen stream, is preferred.

Next, the oil containing the ARA is separated from the biomass by filtration or pressing, preferably at high pressure, and then a final filtration is carried out so as to remove the fine particles of biomass.

It was observed that the level of incorporation of the ARA increased when the size of the biomass particles decreased; it was >90% when for example 90% of the particles had a size <250 μm.

By way of example, it is possible to use a ball mill or a colloidal mill. The parameters to be considered are the duration of grinding, the size of the biomass particles, the grinding temperature, the ratio between the quantities of biomass and of carrier oil.

The duration of grinding has an influence on the size of the particles and the latter is also influenced by the grinding temperature. Consequently, in practice, it is preferable to indicate the size of the particles as a parameter determining the grinding stage. Thus, it is desirable that 90% of the particles have a size <500 μm, preferably that 90% of the particles have a size <300 μm and more preferably still that 90% of the particles have a size <200 μm.

The grinding temperature is chosen at a value greater than the melting point of the carrier oil, and is preferably about 20 to 80° C. To obtain an optimum level of incorporation, a brief grinding may be carried out at a high temperature or a prolonged grinding at a low temperature.

The weight ratio chosen between the biomass and the carrier oil determines the content of ARA of the final oil. Thus, for example, 30 parts of biomass are chosen per 70 parts of carrier oil in order to obtain at least 4.5% of ARA in the converted oil.

The oil used as carrier may be any oil or mixture of oils which can be consumed as human food. An oil or a mixture entering into the composition of the product which it is desired to enrich with PUFA is preferably used. There may be mentioned in particular for an infant formula high oleic acid sunflower oil (HOSFO), sunflower oil (SFO), soya bean oil, palm olein and a medium-chain triacylglycerol (MCT, containing essentially triacylglycerols of saturated C₈-C₁₀ fatty acids).

The next stage of the process consists in separating the spent biomass residue by any customary method such as, for example, pressing, filtration or centrifugation. To this end, a press operating at high pressure is preferably used.

The oil obtained should be made free of fine insoluble particles by fine filtration. This operation may be carried out, where appropriate, by exposing the oil to a mineral adsorbent as a filter aid, for example dicalite.

Finally, the filtered oil is deodorized in order to remove the volatile substances. This may be carried out by any known method provided that moderate conditions are used in order to be gentle with the ARA. There may be mentioned, for example, steam distillation, preferably under vacuum, or molecular distillation.

The oil obtained may be used in food compositions for human consumption as it is or in the form of an emulsion such as, for example, oils, salad dressings or mayonnaise. It may be a constituent of a dietetic milk for teenagers or adults, an infant formula for premature babies, full-term unweaned babies or a follow-on milk for small children.

It may be incorporated into a nutritive or supplemental composition for oral consumption.

It may be incorporated into a pharmaceutical composition for oral, enteral or parenteral ingestion, or for topical, dermatological or opthalmological application.

It may constitute an ingredient for a cosmetic, topical or oral composition.

Finally, it may constitute an ingredient for a pet food, for example a dry or moist food or even a milk.

The biomass residue, after separation of the oil, may be advantageously used in animal feed, particularly for pets.

### EXAMPLES

The examples below illustrate the invention. Therein, the parts and percentages are by weight, unless otherwise stated. The biomass:carrier oil ratio is 3:7.

### Examples 1-6

In these examples, the process parameters and the quality of the oil obtained, before the final deodorization stage, compared with the starting carrier oil (reference 1) are studied. For the grinding, a ball mill is used. The results are summarized in Table 1 below:

TABLE 1

| | | | | | | | Reference |
|---|---|---|---|---|---|---|---|
| Example | 1 | 2 | 3 | 4 | 5 | 6 | 1 |
| Grinding | 50 | 70 | 50 | 30 | 70 | 30 | Carrier |
| temperature | | | | | | | oil* |
| (° C.) | | | | | | | |
| Grinding time | 3 | 5 | 3 | 5 | 1 | 1 | — |
| (min) | | | | | | | |
| Free fatty | 0.13 | 0.13 | 0.14 | 0.12 | 0.17 | 0.15 | 0.14 |
| acids (%) | | | | | | | |
| IUPAC 2.201 | | | | | | | |
| Peroxide value | 4.0 | 4.1 | 3.5 | 3.5 | 4.0 | 3.4 | 2.7 |
| (meq/kg) | | | | | | | |
| AOCS Cd 8b-90 | | | | | | | |
| Unsaponifiable | 8.34 | 9.20 | 8.82 | 8.03 | 8.11 | 7.44 | 6.78 |
| components | | | | | | | |
| (g/kg) | | | | | | | |
| IUPAC 2.401 | | | | | | | |
| ARA (g ARA/ | 4.4 | 4.87 | 4.25 | 4.46 | 3.81 | 3.27 | 0 |
| 100 g oil) | | | | | | | |
| IUPAC 2.304 | | | | | | | |
| Phosphorus | 2 | 1 | 3 | 2 | 2 | 4 | 6 |
| (ppm) | | | | | | | |
| NI C12-1976- | | | | | | | |
| SSOG | | | | | | | |

*The carrier oil is a high oleic acid sunflower oil, TRISUN ™.

The theoretical value for incorporation of 100% of ARA is 5.3% with a biomass:carrier oil ratio of 3:7.

The purity of the crude oil may be qualified by the values below:
Free fatty acids: 0.13-0.17% (TRISUN, carrier oil: 0.14%)
Phosphorus: 1-4 ppm (part per million) (TRISUN: 6 ppm)
Unsaponifiable matter: 7.4-9.2 g/kg (TRISUN: 6.8 g/kg)

In conclusion:
More than 60% of the ARA of the biomass is incorporated into the carrier oil.
The phosphorus content is very low, a few ppm, approximately 100× less than in the case of the crude oil extracted with hexane which was about 500 ppm.

### Examples 7-10

In these examples, the process parameters and the quality of the oil obtained, after the final deodorization stage, during the use of various carrier oils, are studied. In these examples, the grinding of the biomass is carried out using a ball mill.

The characteristics of the oils obtained are compared with the crude oil obtained by extraction with hexane, without refining (reference 2) and compared with the oil obtained by direct pressing, and therefore with no carrier oil (reference 3).

The results are summarized in Table 2 below:

TABLE 2

| | | | | | Refer- | Refer- |
|---|---|---|---|---|---|---|
| Example | 7 | 8 | 9 | 10 | ence 2 | ence 3 |
| Carrier oil | HOSFO* | HOSFO* | MCT | Palm | | |
| | | | | olein | | |
| Grinding | 70 | 30 | 70 | 70 | | |
| conditions: | | | | | | |
| temperature | | | | | | |
| (° C.), | | | | | | |
| Time (min) | 5 | 10 | 5 | 5 | | |
| Free fatty | 0.04 | 0.03 | 0.04 | 0.04 | 0.56 | 0.11 |
| acids (%) | | | | | | |
| Peroxide | 2.0 | 2.3 | 2.3 | 1.3 | 11.5 | 4.8 |
| value | | | | | | |
| (meq/kg) | | | | | | |
| Unsaponifiable | 8.02 | 7.05 | 3.53 | 5.40 | 22.89 | 17.59 |
| matter | | | | | | |
| (g/kg) | | | | | | |
| Phosphorus | 3 | 4 | 4 | 3 | 508 | 17 |
| (ppm) | | | | | | |
| ARA | 4.6 | 4.7 | 4.5 | 4.4 | 39.5 | 39.6 |
| (g ARA/100 g | | | | | | |
| oil) | | | | | | |

*The carrier oil is a high oleic acid sunflower oil (HOSFO).

The results obtained allow the following conclusions:
The grinding temperature and time are linked: a grinding of 10 min at 30° C. gives the same level of incorporation of ARA as a grinding of 5 min at 70° C.
The level of incorporation depends only slightly on the type of carrier oil when the procedure is carried out at the same grinding temperature/time: high oleic acid sunflower oil (4.9% ARA at 70° C./5 min), MCT (5.0% ARA at 70° C./5 min) and palm olein (5.0% ARA at 70° C./5 min).
A very small quantity of phosphorus is obtained compared to that obtained by extraction with hexane, which shows the purity of the final oil.

### Examples 11-14

The examples below show the preparation of an oil containing ARA in the form of triacylglycerols by a process which is gentle with the quality of the ARA by using several routes, without grinding (Example 11) and with various types of grinding equipment (Examples 12-14).

### Materials Used:

Biomass containing 36.3% of oil to 39.5% of arachidonic acid (ARA).

High oleic acid sunflower oil.

Palm olein.

MCT oil.

### Example 11

### Preparation by Contacting with High Oleic Acid Sunflower Oil

### Equipment:

Stirred glass reactor of 1000 ml with a double jacket, linked to a thermostatted bath.

Carver press with a 48×200 mm filtration cartridge.

Thermostatted bell-shaped filter MAVAG 300 ml.

Laboratory deodorizer according to J. HEIDE-JENSEN (JAOCS; Vol. 40, 223-224; 1963) with a 1000 ml round-bottomed flask.

### Procedure:

260 g of high oleic acid sunflower oil and 112 g of biomass are introduced into the reactor. The vessel is placed under vacuum and the air is replaced with nitrogen three times for the inerting. The reactor is then stirred at 50° C. for 2 h, and then the mixture is recovered in a filtration cartridge. The oil is separated from the biomass by pressing. 260 g of oil and 110 g of cake are recovered.

The pressed oil is filtered at 50° C., and then it is deodorized at 180° C., 1 mbar for 2.6 h. 240 g of a clear oil having a neutral odour and a light yellow colour are finally obtained. The ARA content of the oil is determined by gas chromatography (GC) analysis and the level of incorporation of ARA is calculated.

### Example 12

### Preparation by Grinding with Palm Olein in a Ball Mill

### Equipment:

DYNO-MILL type KDL ball mill with 0.3 l grinding vessel, double jacket linked to a thermostatted bath.

Carver press with a 48×200 mm filtration cartridge.

Thermostatted bell-shaped filter MAVAG 300 ml.

Laboratory deodorizer according to J. HEIDE-JENSEN (JAOCS; Vol. 40, 223-224; 1963) with 1000 ml round-bottomed flask.

### Procedure:

130 g of palm olein and 56 g of biomass are introduced into the mill vessel. The vessel is placed in a vacuum and the air is replaced with nitrogen three times for the inerting. 220 ml of glass beads having a diameter of 2 mm are added and the vessel is heated to 65° C. with the aid of a thermostatted bath. The mixture is then ground for 5 min at a temperature of 65 to 75° C., and then the vessel is emptied. The beads are separated from the mixture by filtration on a grid having a diameter of 1 mm, the mixture is recovered in a filtration cartridge and a sample is collected for measuring the size of the particles. The oil is separated from the biomass by pressing. The procedure is repeated a second time.

225 g of oil and 75 g of cake are recovered. The pressed oil is filtered at 50° C., and then it is deodorized at 180° C., 1 mbar for 2.2 h. 210 g of a clear oil having a neutral odour and a light yellow colour are finally obtained. The ARA content of the oil is determined by GC analysis and the level of incorporation of ARA is calculated.

### Example 13

### Preparation by Grinding with MCT Oil in a Ball Mill

The trial described in Example 12 is repeated using MCT oil as a replacement for palm olein. 235 g of oil and 65 g of cake are finally recovered.

The pressed oil is filtered at 50° C., and then it is deodorized at 180° C., 1 mbar for 2.3 h. 220 g of a clear oil having a neutral odor and a light yellow color are finally obtained. The ARA content of the oil is determined by GC analysis and the level of incorporation of ARA is calculated.

### Example 14

### Preparation by Grinding with High Oleic Acid Sunflower Oil in a Colloid Mill

### Equipment:

FRYMA MZ 80 colloid mill.

PADBERG basket centrifuge.

Thermostatted bell-shaped filter MAVAG 300 ml.

Laboratory deodorizer according to J. HEIDE-JENSEN (JAOCS; Vol. 40, 223-224; 1963) with a 1000 ml round-bottomed flask.

### Procedure:

2800 g of high oleic acid sunflower oil and 1200 g of biomass are introduced into the mill vessel. The grinding is carried out by recirculating the mixture in the grinder under an inert atmosphere for 10 minutes at a temperature of 40 to 70° C. The mixture is recovered and a sample is collected for measuring the size of the particles. The oil is separated from the biomass with the aid of the basket centrifuge. 2400 g of oil and 1400 g of cake are finally recovered.

200 g of centrifuged oil are filtered at 50° C., and then it is deodorized at 180° C., 1 millibar for 2 hours. 190 g of a clear oil having a neutral odour and a light yellow colour are finally obtained. The ARA content of the oil is determined by GC analysis and the level of incorporation of ARA is calculated. The results are indicated in Table 3 below:

TABLE 3

| | Size of the particles | g ARA | % level of |
|---|---|---|---|
| | (Malvern Mastersizer) | in 100 g | incorporation |
| Example | micron D (v, 0.9)* | oil | of ARA |
| 11 | 3000 | 3.5 | 66.1 |
| 12 | 75 | 5.0 | 94.3 |
| 13 | 60 | 5.0 | 94.3 |
| 14 | 115 | 5.25 | 99.0 |

*Micron, D (v, 0.9): means that 90% by volume of the particles have a diameter of less than D.

### Example 15

### Incorporation of DHA into the High Oleic Acid Sunflower Oil

### Equipment:

FRYMA MZ 80 colloid mill.

PADBERG basket centrifuge.

Thermostatted bell-shaped filter MAVAG 300 ml.

Laboratory deodorizer according to J. HEIDE-JENSEN (JAOCS; Vol. 40, 223-224; 1963) with a 1000 ml round-bottomed flask.

### Procedure:

The procedure of Example 14 is repeated by treating 1200 g of a biomass containing 25% of an oil with a DHA content of 40%. 2500 g of oil with a DHA content of 3.5% are recovered, which oil is deodorized.

### Examples 16-17

An infant formula for premature babies enriched with ARA is prepared from the oil prepared by the process of Examples 12 or 13 and there are added thereto other oils, for example in the proportions indicated in Table 4 below, proteins, where appropriate hydrolyzed, carbohydrates and where appropriate vitamins and trace elements.

TABLE 4

| | Example 16 | Example 17 |
|---|---|---|
| Oil of Example 12 | 4 | — |
| Oil of Example 13 | — | 4 |
| Fish oil | 1.5 | 1.5 |
| MCT oil | 27 | 25 |
| Soya bean oil | 23 | 23 |
| Palm olein | 44.5 | 35 |
| High oleic acid | — | 11.5 |
| sunflower oil | | |
| Total | 100 | 100 |

### Examples 18-19

An infant formula for full-term unweaned babies enriched with ARA is prepared from the carrier oil prepared by the process of Examples 13 or 14 and there are added thereto other oils, for example in the proportions indicated in Table 5 below, proteins, where appropriate hydrolyzed, carbohydrates and where appropriate vitamins and trace elements.

TABLE 5

| | Example 18 | Example 19 |
|---|---|---|
| Oil of Example 13 | 4.5 | — |
| Oil of Example 14 | — | 7 |
| Fish oil | 1.5 | 1.5 |
| Coconut oil | 20 | 27.5 |
| Soya bean oil | 20 | 20 |
| Palm olein | 54 | 44 |
| Total | 100 | 100 |

### Examples 20-21

A follow-on milk for small children enriched with ARA is prepared from the carrier oil prepared by the process of Example 12 or enriched with DHA from the carrier oil prepared by the process of Example 15, and there are added thereto other oils in the proportions indicated in Table 6 below, proteins, where appropriate hydrolyzed, carbohydrates and where appropriate vitamins and trace elements.

TABLE 6

| | Example 20 | Example 21 |
|---|---|---|
| Oil of Example 12 | 4 | — |
| Oil of Example 15 | — | 7 |
| Fish oil | 1.5 | — |
| Palm kernel oil | 27 | — |
| Coconut oil | — | 19 |
| Soya bean oil | 23 | — |
| Rapeseed oil | — | 30 |
| Palm olein | 44.5 | 44 |
| Total | 100 | 100 |

### Example 22

A liquid milk enriched with DHA in an amount of 1% of DHA in the fatty phase is prepared in the following manner:

A whole milk containing 3.92% of fat and 8.58% of solids-not-fat and a low-fat milk containing 0.05% of fat and 9% of solids-not-fat are pasteurized separately by treating them at 87° C. for 12 s.

34.69 kg of whole milk and 160.26 kg of low-fat milk, cooled to 15° C. are then mixed, and then a premix of 1.08 kg of oil obtained according to Example 15 (high oleic acid sunflower oil, containing 3.5% of DHA), 1.08 kg of soya bean oil and 1 g of vitamin E heated to 50° C. is incorporated into this mixture by means of a colloid mill.

### Sterilized Product:

After heating to 80° C. in a plate exchanger, the liquid is UHT sterilized at 148° C. for 5 s. After cooling at 78° C., it is homogenized in two stages, at 200 bar, and then at 50 bar; it is cooled to 20° C. and it is aseptically packaged in carton-type packaging which has been previously sterilized, the homogenization, cooling and filling stages taking place aseptically.

### Pasteurized Product:

The liquid is heated at 72° C. for 15 s in a plate exchanger; it is homogenized in two stages at 200 bar, and then at 50 bar; it is cooled to 4° C. and it is packaged in carton-type packaging.

### Example 23

As a nutritional supplement, an oil prepared according to Example 12, 13 or 14 containing ARA or an oil prepared according to Example 15 containing DHA, is encapsulated in an amount of 500 mg of oil in gelatine capsules.

1-19. (canceled)

20. A stable oil for incorporation in a food, nutritional, pharmaceutical or cosmetic product, the stable oil made by a process comprising combining a carrier oil and one or more long-chain polyunsaturated fatty acids from a biomass obtained from the culture of a microorganism, in the form of triacylglycerols, wherein the long-chain polyunsaturated fatty acids are incorporated into the carrier oil such that at least 60% by weight of the long-chain polyunsaturated fatty acids present in the biomass are present in the carrier oil but that less than 10% of phosphorus that is present in the biomass is present in the carrier oil, so that the carrier oil does not require purification prior to use and wherein the stable oil contains no more than 10% by weight of the long-chain polyunsaturated fatty acids in the form of triacylglycerols.

21. The stable oil according to claim 20, in which the long-chain polyunsaturated fatty acid is arachidonic acid, dihomogammalinolenic acid, eicosapentaenoic acid or docosahexaenoic acid.

22. The stable oil according to claim 21, in which the long-chain polyunsaturated fatty acid is arachidonic acid.

23. The stable oil according to claim 21, in which the long-chain polyunsaturated fatty acid is docosahexaenoic acid.

24. The stable oil according to claim 20, in which the carrier oil is high oleic acid sunflower oil (HOSFO), sunflower oil (SFO), soya bean oil, palm olein or a medium-chain triacylglycerol (MCT, containing essentially triacylglycerols of saturated C₈-C₁₀ fatty acids).

25. A process for preparing a stable oil for incorporation into a composition of a food, nutritional, pharmaceutical or cosmetic product, the process comprising bringing a carrier oil into contact with a biomass obtained from the culture of a microorganism containing one or more long-chain polyunsaturated fatty acids, so as to transfer the long-chain polyunsaturated fatty acid(s) in the form of triacylglycerols to the carrier oil and form a biomass residue, separating the carrier oil containing the fatty acid(s) from the biomass residue, and then deodorizing the separated carrier oil to obtain the stable oil, wherein the stable oil contains no more than 10% by weight of the long-chain polyunsaturated fatty acids in the form of triacylglycerols.

26. The process according to claim 25, which further comprises grinding the biomass prior to contact with the carrier oil to reduce particle size in order to break cell walls of the microorganisms and to thereby increase contact surface area between the carrier oil and the biomass.

27. The process according to claim 25, which further comprises contacting the biomass under high pressure with the carrier oil, and separating the oil containing the long-chain polyunsaturated fatty acid from the biomass residue by pressing and filtration.

28. The process according to claim 25, which further comprises grinding the biomass in the presence of the carrier oil under an inert atmosphere.

29. The process according to claim 25, which is carried out under a nitrogen atmosphere and in the presence of one or more tocopherols in an amount sufficient to protect the LC-PUFAs from oxidative degradation.

30. The process according to claim 25, wherein the deodorizing is achieved by molecular distillation or steam distillation, optionally assisted by vacuum.

31. The process according to claim 25, which further comprises a fine filtration of the stable oil to remove fine biomass particles.

32. The stable oil obtained by the process of claim 25.

33. The product of claim 25 wherein the product is an infant foodstuff, a nutritional composition, a cosmetic composition, or a foodstuff.

34. A product containing a stable oil according to claim 20.

35. The product of claim 34 wherein the product is an infant foodstuff, a nutritional composition, a cosmetic composition, or a foodstuff.

36. An animal feed containing the biomass residue obtained by the process according to claim 25.
